(19) United States
(12) Patent Application Publication
(10) Pub. No.: US 2009/0029447 A1
(43) Pub. Date: Jan. 29, 2009

(54) Process for removing mercury from air or water

(75) Inventors: Richard Huffman Squire, South Charleston, WV (US); Jay Michael Wiedermann, Powellton, WV (US); Ranjith Arachchige Munasinghe, Charleston, WV (US); Milson Jude Luce, Smithers, WV (US); Richard C. Schoening, Charleston, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(73) Assignee: West Virginia University

(21) Appl. No.: 11/881,308

(22) Filed: Jul. 26, 2007

(51) Int. Cl. A62D 3/02; C12M 1/00

(52) U.S. Cl. 435/262.5; 435/303.1

(57) ABSTRACT

The present invention provides for the removal of mercury utilizing Mer proteins. After the Mer proteins have been expressed and isolated, or alternatively using the native Mer protein complex, either can be used as a chelating agent to remove mercury from any aqueous mercury containing environment. The Mer protein may be used in a solution to bind mercury where the solution contains a Mer proteins may be reversibly attached to a removal apparatus as a Mer ligand. The Mer complex may also be used within a bioreactor to bind and reduce Hg⁽²⁺⁾. The bioreactor may have any competent bacteria capable of producing a Mer complex attached within the bioreactor also containing an inlet and an outlet. In addition, the Mer complex may be enhanced within Mer competent bacteria by a method of enhancement that comprises the creation of a baseline of Mer competent bacterial growth, creating a stock of Mer competent bacteria growing at the baseline level and then adding baseline bacteria to successively higher levels of Hg until the bacteria can grow in 1 mM Hg. The advantage of the Mer proteins is that the mercury binding is exclusive in regards to other metals.

### CROSS-REFERENCE TO RELATED APPLICATIONS

This application claims priority from application No. 60/833332

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

### BACKGROUND OF THE INVENTION

1. Field of Invention

This invention relates to the removal of mercury from aqueous or gaseous environments by the MerP protein and Mer bacterial protein complex.

2. Description of the Prior Art

Much of the coal produced is burned to generate electricity. Unlike an organic contaminant that can be destroyed in an environmentally acceptable manner by burning, metallic components cannot be destroyed. Burning coal containing mercury releases mercury in its elemental form. In most animals mercury is a cumulative lipophilic poison that aggregates in materials such as fatty tissue. History contains numerous examples of toxic human exposure to mercury. Regulations that become effective in 2007 require a significant reduction in mercury emissions. Power plants have varied designs that result in process streams unique to that particular plant. In addition, changing operating conditions and the types of coal burned can further compound the variability of process streams. These parameters provide large variability from plant to plant. Therefore making the capture of mercury and the reduction of Hg⁽⁺²⁾ and/or organomercury to non-toxic Hg⁽⁰⁾ and make capture quite difficult or impossible. In addition, a general inability to remove Hg⁽⁰⁾ as opposed to Hg⁽⁺²⁾ further complicates the removal of mercury. Also hindering the removal of mercury is its low concentration such that large amounts of current, non-specific binding chemical ligands are required,

Removal of mercury from aqueous sources has been a continual problem for years. Whether from power plants, water treatment centers, mercury-cell electrolysis plants or coal containing solutions there is an ongoing need for mercury removal. According to U.S. Environmental Protection Agency power plants are one of the main emitters of mercury at 48 tons a year in the United States. In places where coal is used for electricity production, the coal fines, along with water that has been in contact with the coal, are stored in coal impoundment ponds. Since mercury is present in the coal and water, the ponds continually grow larger as no technology is currently available for accurate removal of the mercury without considerable expense. While there is some debate as to the form of mercury in coal, there is no doubt that mercury is present. Recent measurements of locally obtained fine coal particle samples from impoundment ponds by researchers at West Virginia University-Institute of Technology suggest the presence of 10 to 15 parts mercury per billion; however, mercury levels in coal may vary geographically, being measured as high as 70 parts mercury per billion in some local coal. Current technologies utilize expensive non-specific ligands that also capture numerous other metals requiring an excessive amount of the ligand. When this process is carried out, an irreversible binding complex is formed, eliminating any chance to use the ligands multiple times.

Ligands which exclusively bind to mercury are beneficial. These ligands are not bound to similar metals so a lesser quantity may be used to provide the same effect as that of a greater quantity of non-specific ligands. A series of naturally occurring (native) bacterial Mer proteins chelate mercury exclusively, then pass the mercury to a reductase protein via a transport protein to reduce mercury from an Hg⁽⁺²⁾ to Hg⁽⁰⁾ in living bacteria (100). This Mer family of proteins has evolved over time to allow the bacteria to survive in a toxic mercury environment. Several of the species have been isolated from the coal impoundment ponds. Laboratory work has established that low levels of mercury can be captured by these Mer-type proteins.

### BRIEF SUMMARY OF THE INVENTION

The present invention pertains to the capture of mercury by reacting the mercury with a biochemical protein which would function as a chemical ligand to form a complex for removal of the mercury. The Mer proteins found in native bacteria are efficient mercury removal proteins for the binding, reduction, and elimination of mercury. These proteins are able to exclusively bind mercury in environments containing other metals to which non-exclusive ligands would be irreversibly bound. The MerP proteins are the preferred protein used as the complexing agent. The native bacteria protein complex may be the most practical.

The present invention can have the additional aspect of using the Mer proteins in a removal apparatus where the Mer protein is attached to the removal apparatus and exposed to a sample containing Hg⁽²⁺⁾ for the binding of the Hg⁽²⁺⁾ to the removal apparatus.

A further aspect of the present invention is the use of a Mer protein complex within competent bacteria to bind and reduce Hg⁽²⁺⁾. The bacteria can be used within a bioreactor for the reduction of mercury from different environments.

The present invention additionally includes the production of the Mer proteins using a mixed population of many native bacteria strains and/or transformed bacteria such as E. coli. These techniques enable proteins and protein complexes to be produced relatively easily and inexpensively for use as chemical ligands and for other functions.

Another aspect of the present invention is the cost-effective process to reduce the mercury content of mercury containing solutions and/or their effluent. The present invention is especially effective in the removal of mercury from coal impoundment ponds. The invention specifically complexes only mercury with a protein from the Mer family, specifically MerP or a protein complex containing MerP. The resulting cost of the MerP complex removal is significantly less than with standard reagents. In addition the present invention includes the ability of the removed mercury to be converted to Hg⁽⁰⁾ for future use.

In addition the present invention details a method of enhancement to produce competent bacterial colonies with more efficient Hg binding and reduction than native colonies.

### BRIEF DESCRIPTION OF THE SEVERAL VIEWS OF THE DRAWING

FIG. 1 is the Mer protein complex.

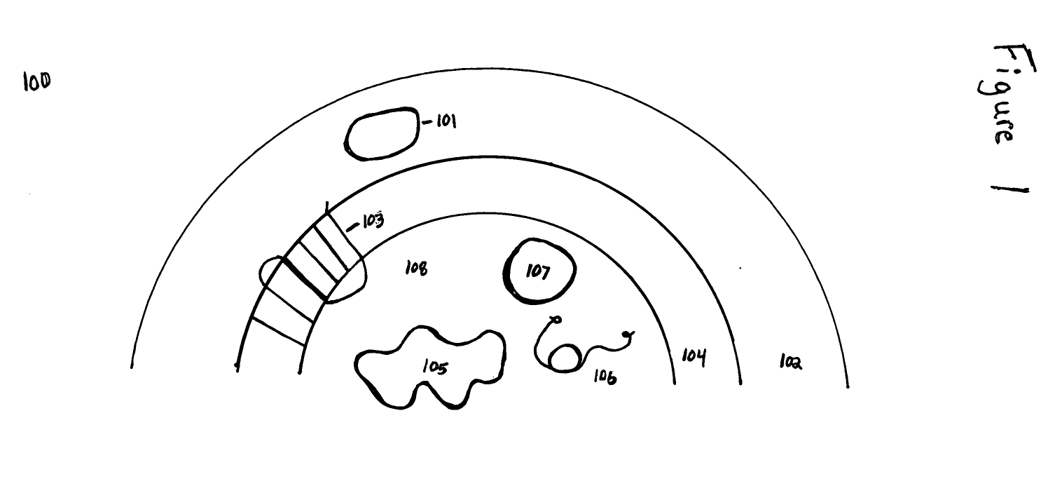

FIG. 2 is a continuous mercury removal process.

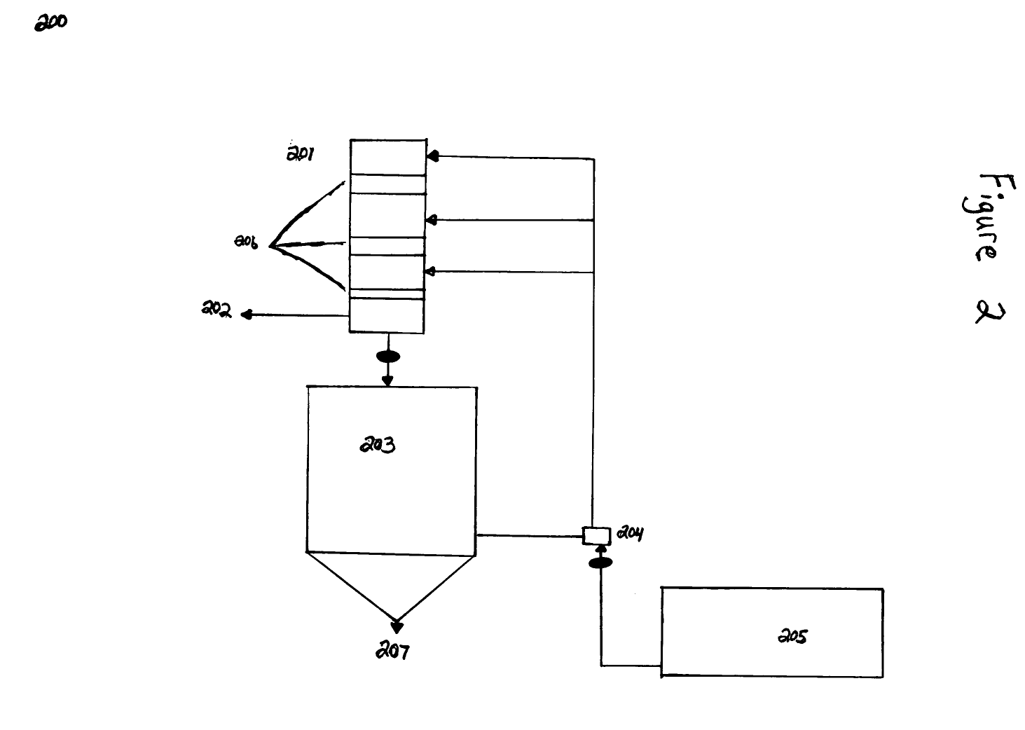

### DETAILED DESCRIPTION OF THE INVENTION

The Mer proteins can be any of the recognized amino acid sequences for each of the different Mer proteins and the conservative variants thereof. Conservative variants are amino acid sequences that may differ from the normal sequence; however, the tertiary protein still has virtually the same binding features as the normal protein sequence. Conservative variants are often a replacement of one or more amino acids with those of similar properties on the side chain. A Mer complex, as used in this application, is one or more Mer proteins capable of the capture and reduction of mercury when utilized in concert such as by competent bacteria 100.

The MerP protein 101 is a mercury ion-binding and mercury scavenger protein found within the periplasmic space 102. MerP has 537 different sequences with many of these sequences being truncated. In this application, a MerP protein has a CAAC Hg⁽⁺²⁾ binding site in a linear bicoordinate complex with high binding constant. The MerT protein 103 is a mercuric transport integral membrane protein found within the cell membrane 104. There are 388 known MerT sequences. An important feature of the bacterial mercury detoxification system lies with the transport protein, MerT with binding sequence CCAA that binds Hg⁽⁺²⁾ exclusively, thereby further increasing the mercury selectivity. The MerA protein 105 is an Hg⁽²⁺⁾ reductase with 907 different known sequences for the protein. The MerR 106 is a MERR transcription regulator with 7604 different sequences, most of which are truncated. The MerB 107 protein is an organomercury lyase with 136 different sequences. The MerA, Mer R, and MerB proteins can all be found within the cytoplasm 108. The Mer proteins can be any of the sequences known to one with ordinary skill in the art or a constructive variant thereof as long as the sequence or the constructive variant produces a functional Mer protein for mercury binding and/or reduction.

The Mer protein can be found in many bacterial species and can also be transformed and expressed in E. coli or any other competent bacteria. Some bacteria found naturally having Mer proteins expressed are Bacillus megaterium, Bacillus cereus, Clostridium butyricum, Staphylococcus aureus pI258, Streptomyces lividans, Streptomyces pRJ28, Exiguobacterium sp., Pseudomonas sp. ED-23, Pseudomonas stuizeri OXpPB, Serratia marcescens DU1358, Pseudomonas aeruginosa Tn501, Alcaligenes pMER610, Shigella flexneri Tn21, Pseudomonas sp. ADP, Xanthomonas campestris Tn5044, Xanthomonas sp. Tn5053, Pseudomonasfluorescens, Shewanella putrefaciens pMERPH, Thiobacillus ferrooxidans, Pseudoalteromonas haloplanktis, Acidithiobacillus ferrooxidans SUG 2-2, Acidithiobacillus ferrooxidans MON-1. Expressed proteins can be isolated from the bacteria by standard biochemical techniques such as affinity chromatography, other chromatography techniques, and other standard techniques within microbiology.

After the Mer protein has been expressed and isolated, or alternatively using the native Mer protein complex, either can be used as a chelating agent to remove mercury from any mercury containing environment. The MerP protein may be used in a solution to bind mercury where the Mer protein complex may be reversibly attached to a removal apparatus. The reaction of the Mer proteins, specifically the MerP proteins or Mer protein complex, with mercury to form a chelate (chemical complex) in the lab has been established using actual industrial coal samples and other industrial solutions/slurries. The quantitative aspects indicate that the reaction is so fast in very dilute solutions (ppb) that it is difficult to detect low levels of mercury using normal analytical methods such as atomic absorption. In higher concentrations (ppm) about 0.008 mM (milliMolar) Hg⁽⁺²⁾ is converted to Hg⁽⁰⁾ per hour by the protein complex. In a MerP chelating process, the removed mercury can be collected at a high concentration for conversion to elemental mercury by MerA or chemical reduction. With the appropriate design either MerP or the Mer protein complex can be used to remove dilute mercury from water or air.

A typical example of a process to remove mercury from an aqueous environment is the attachment of the MerP protein or Mer complex to a removal apparatus designed to provide the necessary residence time to react with the volume of effluent desired from a coal slurry pond. A removal apparatus can be beads, a support column, a column packed with the Mer protein or Mer complex, fiber mats or any other means for attaching the Mer protein or Mer protein complex. There are numerous attachment possibilities for either the Mer protein or Mer complex known to one skilled in the art such as protein attachment to a removal apparatus by any conventional ligand attachment method such as a Strep-Tag from Sigma-Genosys. The proper operation is to use standard methods so that the protein or protein complexes do not pack too tightly so as to allow uniform diffusion through the bed or column. Redistributors are standard in this technology as well as angled bed supports with multiple diffusion supports that maintain a low pressure drop through the beds. Back flushing is also a common practice to insure proper distribution. The volume of the effluent may be increased or decreased by changing the cross section and/or volume of the removal apparatus for use in different sized ponds. Existing technology such as an in-line mercury monitor to detect mercury in solution can be used to detect when the binding of the MerP and mercury in the removal apparatus nears reaction completion. When a first removal apparatus is nearly completely reacted, the mercury containing effluent can be switched to a second removal apparatus while the first is regenerated. After the first apparatus is removed for regeneration the second apparatus is brought on-line by a means of exchange which can be any means standard to one skilled within the art. The regeneration may be performed by a means to regenerate. The means to regenerate can be one of two methods: either a very specific concentration of a salt such as guanidine hydrochloride, or raising the protein to an elevated temperature between about 55° C. to about 65° C. The salt method of regeneration can be performed by using an amount of guanidine hydrochloride that can vary from about 0.35M to about 2.90M to denature MerP in steps. The steps denature a portion of the protein at a time. The regeneration step performs two functions. The first function is the release of the mercury from the Mer protein or Mer complex attached to the removal apparatus where the mercury is released into a concentrated solution. The concentrated solution can then be reduced from Hg⁽⁺²⁾ to Hg⁽⁰⁾ by a means of reduction such as Mer complex capable bacteria or chemical treatment to form an insoluble compound. The means of reduction will produce elemental mercury Hg⁽⁰⁾ which is then collected by either phase separation or centrifugation, depending on the concentration. The second function of regeneration is to allow the MerP protein to be renatured quickly either by dropping the temperature or the salt concentration slowly with appropriate buffering so the column can be brought back on-line in an expeditious manner.

A potentially more cost-effective alternative to current mercury removal techniques is a continuous bioreactor 200 packed with living colonies of mixed native and/or transformed bacteria capable of producing a Mer protein complex. The bioreactor may additionally include enhanced bacteria. The mercury removal activity of the native bacteria is enhanced through culturing in sequentially increasing levels of mercury. The enhancement of the bacteria can be achieved by a progressive increasing of Hg content in a growth media during successive generations of bacterial growth. A growth media can be any conventional bacterial growth media such as one containing phenol red mannitol, litmus milk, tryptic soy broth, urea, nutrient broth and LB broth, a combination of tryptone, yeast extract, and sodium chloride, pH 7.0 with no mercury. Mer competent bacteria can then be transferred into a growth media with varying levels of Hg added such as three different medias with 0.001, 0.005, and 0.01 nM Hg⁽⁺²⁾ respectively. The bacteria are then incubated 24 hours and checked for bacteria growth. The colony with both the highest Hg concentration and bacterial growth can be used as a baseline. From that baseline fresh growth media with the baseline level of Hg can be inoculated every 24 hours for a week to create stock bacteria able to grow at the baseline. Using the same procedure as above, the mercury concentration can then be raised to successively higher levels: 0.003 mM, 0.01 mM, 0.05 mM, 0.1 mM, 0.2 mM, 0.4 mM, 0.6 mM, and finally 1 mM Hg⁽⁺²⁾ while adding in stock samples and freezing samples for recovery, if needed. Following the freezing practice, enhanced bacteria can be kept indefinitely and used at will. The method of enhancement would also work for the increased ability of bacteria to eliminate any other materials the bacteria has the ability to eliminate in normal conditions.

The bioreactor could be comprised of a filter with the filter having an inlet 201 and an outlet 202 for effluent to enter and exit and a means to adhere the bacteria within the filter. The means of attachment for the bacteria within the filter could be beds such as cellulose or fiber mats or any other means known to one skilled in the art. The velocity of the effluent may be increased or decreased by changing the cross section and/or volume of the filter. The living bacteria in the filter could receive nutrients such as Luria-Bertani medium from a nutrient tank 203 wherein the nutrients can be added to the filter through a means to transfer nutrients such as a pump 204 or other conventional means known to one skilled in the art. In addition, the bioreactor can include a bacterial supply 205 for the ability to re-supply the bioreactor with Mer competent bacteria. The bacterial supply can be a holding tank or any other supply mechanism known to one skilled in the art. The bacterial supply has additional Mer complex competent bacteria growing within for addition to the bioreactor. The bacterial supply can also have a means to transfer the bacteria from the bacterial supply to the bioreactor. The means to transfer can be a pump or any other means of transfer known to one skilled in the art. The bioreactor may also include one or more protein supports 206 which can be made of any material known to one skilled in the art such as fiber or cellulose mats, wood chips, peat moss, or any other standard material. The bioreactor can also provide a controlled environment for the bacteria to optimize the mercury reduction. Conditions such as temperature and pH can be monitored by a means to monitor conditions such as thermocouples placed appropriately in the bed to insure temperature gradients are noted. The means to adjust the temperature, especially in winter can be a heat exchanger in which the warm, mercury free exit water heats the incoming effluent-laden stream. Additional heat input can be provided by standard heating techniques such as electric or natural gas heaters or bottled propane heaters. The pH can also be monitored by a standard pH meter for aqueous solutions and adjusted by standard caustic/acid additions common to those skilled in the art of wastewater treatment. However, in a large coal impoundment pond the pH is not expected to vary much. In an industrial stream some pretreatment of the effluent to be treated may be necessary using a means of pretreatment standard in the chemical industry. A bioreactor could be used with liquid or gas streams containing dilute amounts of mercury with the emphasis on streams that have contacted coal or byproducts from coal burning power plants. The bioreactor may also have a means for the collection of metal mercury 207 such as phase separation and centrifugation. Alternatively, a standard chemical waste water treatment system containing only the Mer protein complex can be used for removing mercury from large volumes of contaminated water.

Each of the above examples could be installed in the byproduct exit stream of either a bag house or an electrostatic precipitator or a flue gas desulfurizer scrubber unit. With some stream modification to insure the protein or living bio colony is not destroyed, the process could also be effectively used to remove mercury from effluent streams so these streams could be used for the manufacture of gypsum or concrete products without mercury present. In addition, the processes could be further modified so that they may be installed on the flue gas stream after various units (ESP, FGD, etc) have performed their tasks and preferably just prior to the exit stack. A modest amount of dilution air for temperature control and/or other oxidizing agents such as aluminum foil can be added to insure Hg⁽⁰⁾ is fully oxidized. The Hg⁽⁰⁾ can then be oxidized to Hg⁽⁺²⁾ by an oxidizer such as a dilute permanganate solution scrubber or a column packed with aluminum wool or thin aluminum strips to increase the metal surface area and the mercury can be quantitatively captured by the Mer protein and/or Mer bioreactor colony. Aluminum acts as a solid state battery for the oxidation of Hg. A metal to metal electron transfer at ambient temperatures begins almost instantaneously.

Mercury metal can be captured from the reduction of Hg⁽⁺²⁾ to Hg⁽⁰⁾. In the bioreactor the Hg⁽⁰⁾ will accumulate as mercury metal removed by phase separation and centrifugation. Insoluble mercury compounds can also be made using standard technology such as hydroxide, chlorides or sulfides.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A bioreactor comprising a filter wherein said filter contains bacteria capable of producing a Mer complex, an inlet, an outlet, and a means to adhere the bacteria within said filter.

2. The bioreactor of claim 1 further comprising one or more protein supports.

3. The bioreactor of claim 1 further comprising a nutrient tank and a means to transfer nutrients from said nutrient tank to said filter.

4. The bioreactor of claim 1 further comprising a bacteria supply reservoir and a means to transfer bacteria from said bacteria supply reservoir to said filter.

5. The bioreactor of claim 1 wherein said bacteria are enhanced bacteria.

6. The bioreactor of claim 5 wherein said bacteria are enhanced by adding Mer complex competent bacteria to a plurality of growth media wherein said growth media have varying levels of Hg⁽⁺²⁾, incubating said bacteria, creating a baseline from bacterial colonies growing within the highest level of Hg within said plurality of growth media, using said baseline to grow a stock of said Mer competent bacteria then adding said stock bacteria to successively higher Hg⁽⁺²⁾ levels until said bacterial colonies are able to grow in 1 mM Hg⁽⁺²⁾ growth media.

7. The bioreactor of claim 1 further comprising a means to monitor the conditions of said filter, a means to adjust the pH of said filter and a means to adjust the temperature of said filter.

8. The bioreactor of claim 1 further comprising a means to back flush said filter.

9. The bioreactor of claim 1 further comprising oxidizing agents.

10. The bioreactor of claim 1 further comprising a means to collect metal mercury.

11. The bioreactor of claim 1 wherein an effluent is pretreated by a means of pretreatment before said effluent is added to said bioreactor.

12. A method to remove exclusively Hg⁽²⁺⁾ from a sample comprising a removal apparatus wherein said removal apparatus has a MerP protein attached by a means of attachment and said removal apparatus is exposed to a sample containing Hg⁽²⁺⁾ wherein said Hg⁽²⁺⁾ binds to said MerP.

13. The method of claim 12 further comprising the oxidization of a sample containing Hg⁽⁰⁾ to Hg⁽⁺²⁾ using aluminum metal with a high surface area for said MerP binding.

14. The method of claim 13 further comprising the reduction of Hg⁽²⁺⁾ to Hg⁽⁰⁾ by releasing bound Hg⁽²⁺⁾ from said removal apparatus by regeneration wherein said Hg⁽²⁺⁾ is released into a concentrated solution containing a means of reduction.

15. The method of claim 14 wherein said means of reduction is a Mer complex.

16. The method of claim 15 wherein said means of reduction is a Mer complex in a Mer competent bacteria.

17. The method of claim 16 wherein said means of reduction is a chemical treatment.

18. The method of claim 14 wherein said sample is a liquid sample.

19. The method of claim 14 wherein said sample is a gas sample said method further comprising a means to control the temperature and a means to add oxidation agents.

20. The method of claim 14 further comprising a means to monitor said removal apparatus for the binding of MerP to Hg⁽²⁺⁾.

21. The method of claim 20 further comprising a means to exchange a 1^{st} removal apparatus wherein said 1^{st} removal apparatus has a binding of MerP to Hg⁽²⁺⁾ within a desired amount wherein said first removal apparatus is replaced with a 2^{nd} removal apparatus.

22. The method of claim 21 further comprising a means to regenerate said 1^{st} removal apparatus.

23. A method of enhancing Mer competent bacteria comprising adding Mer complex competent bacteria to a plurality of growth media wherein said growth media have varying levels of Hg, incubating said bacteria, creating a baseline from bacterial colonies growing within the highest level of Hg within said plurality of growth media, using said baseline to grow a stock of said Mer competent bacteria then adding said stock bacteria to successively higher Hg⁽⁺²⁾ levels until said bacterial colonies are able to grow in 1 mM Hg growth media.

24. A method of oxidizing Hg⁽⁰⁾ to Hg⁽⁺²⁾ wherein said Hg⁽⁰⁾ is exposed to aluminum metal with a high surface area.

25. The method of claim 24 wherein said aluminum metal is a column packed with aluminum wool or thin aluminum strips to increase the metal surface area.

26. The method of claim 25 at ambient temperatures.
